(19) United States
(12) Patent Application Publication
(10) Pub. No.: US 2024/0261349 A1
(43) Pub. Date: Aug. 8, 2024

(54) Pharmaceutical Complex for Radioprotection, Preparation Method, and Applications thereof

(71) Applicant: Zhejiang University, Hangzhou (CN)

(72) Inventors: Min ZHOU, Hangzhou (CN); Dongxiao ZHANG, Hangzhou (CN); Danni ZHONG, Hangzhou (CN); Jian HE, Hangzhou (CN)

(21) Appl. No.: 18/606,877

(22) Filed: Mar. 15, 2024

Related U.S. Application Data
(63) Continuation of application No. PCT/CN2021/143924, filed on Dec. 31, 2021.

(30) Foreign Application Priority Data
Sep. 15, 2021 (CN) 202111082398.4

(51) Int. Cl. A61K 35/748; A61K 9/16; A61K 9/19; A61K 31/661; A61P 39/00

(52) U.S. Cl. CPC A61K 35/748; A61K 9/1688; A61K 9/19; A61K 31/661; A61P 39/00

(57) ABSTRACT

A pharmaceutical complex for radioprotection is disclosed, as well as its preparation method and applications. Within the pharmaceutical complex, the binding between Amifostine and natural microalgae is driven by osmotic pressure. The complex is gradually degraded in the gastrointestinal tract after oral administration. The degradation of the complex makes it thoroughly cover the intestine and slowly release the drug, thereby increasing the drug concentration in the small intestine, sufficiently exerting radioprotective effects on the intestinal tissues, and avoiding systemic toxicity to the gastrointestinal tract. Additionally, the pharmaceutical complex can function as supplementary nutritional substances and regulate intestinal inflammation. The excellent oral safety, feasible preparation, simple storage, and intake of the complex prompt absorption efficiency and oral safety settle the existing issues of drug preparation technologies. The pharmaceutical complex offers wide application prospects in the field of intestinal radioprotection.

### CROSS REFERENCE OF RELATED APPLICATIONS

This application is a continuation of International Patent Application No. PCT/CN2021/143924, filed on 31 Dec. 2021, entitled “Pharmaceutical Complex for Radioprotection, Preparation Method, and Applications thereof” which claims foreign priority of Chinese Patent Application No. 202111082398.4, filed Sep. 15, 2021 in the China National Intellectual Property Administration (CNIPA), the entire contents of which are hereby incorporated by reference in their entireties.

### FIELD OF THE INVENTION

The invention belongs to the technical field of drug preparation, specifically relating to a pharmaceutical complex for radioprotection, as well as its preparation method and applications.

### BACKGROUND

Malignant tumors are one of the diseases that seriously threaten human life and health. Surgery, chemotherapy, and radiotherapy are the mainstay against cancer. Applied to more than half of the patients with malignancy, radiotherapy (RT) plays a critical role in the treatment of multiple tumors. Nevertheless, the ionizing radiation in radiotherapy may inevitably damage the healthy tissues adjacent to the tumor, which affects the functionality of normal tissues or organs. In the radiotherapy of abdominal/pelvic solid tumors, the small intestine with a large organ volume and high radiation sensitivity is extremely vulnerable to radiation to cause radiation-induced intestinal injury (or radiation enteropathy). The intestinal injury induced by radiotherapy results in gastrointestinal dysfunction, such as intestinal epithelial renewal disorder, inflammatory cell infiltration, and dysbiosis of the intestinal microbiota, thereby causing a series of intestinal toxic symptoms (e.g., diarrhoea, vomiting, intestinal bleeding, intestinal perforation, severe infection, and even death). The incidence of radiation-induced injury in patients treated with pelvic or abdominal radiotherapy each year is estimated as high as 60-80% in the United States (Hauer-Jensen, M. et al., Nat. Rev. Gastroenterol. Hepatol. 11, 470-479 (2014).), severely endangering the quality of life of patients. Therefore, it is of great significance to develop drugs, pharmaceutical complexes or drug preparations of the prevention of radiation-induced intestinal injury (i.e., intestinal radiation protection).

Amifostine (3-aminopropylamine ethylthiophosphate, also known as Amifostine or Amifostine, WR-2721, AMF, Amifostine) is a normal tissue-selective radioprotectant approved by the Food and Drug Administration (FDA) for clinical applications. As a prodrug, AMF can be hydrolyzed by intracellular alkaline phosphatase to WR-1065, an active metabolite that protects cells from radiation damage through mechanisms such as scavenging free radicals. AMF exerts a selective protective effect on normal tissues without affecting the anti-tumor effects. The protective mechanism is that normal tissues have higher alkaline phosphatase activity, higher pH (favorable for alkaline phosphatase activity), and better vascular permeability than tumors, thereby enriching more WR-1065 than tumors to produce a specific protective effect (Smoluk, G. D. Et al., Cancer Res. 48, 3641-3647 (1988).). The disadvantage of AMF is also obvious. Firstly, intravenous administration is a traditional delivery route for AMF. However, the rapid clearance and metabolism of AMF from the blood circulation make it hard to reach a sufficient effective concentration in intestinal tissue; increasing intravenous dose can lead to higher blood drug concentration, resulting systemic adverse reactions such as hypotension, nausea, and vomiting (Praetorius, N. P. & Mandal, T. K. J. Pharm. Pharmacol. 60, 809-815 (2008).). Secondly, direct oral administration may result in partial deactivation of AMF due to the low pH of gastric acid, greatly reducing the amount of drug that enters the intestin and failing to protect the intestines effectively (Pamujula, S. E. Al., Int. J. Radiat. Biol. 84, 900-908 (2008).). Therefore, orally delivery of AMF through drug delivery carriers arises as a promising perspective for clinical application of AMF. Currently, the majority of current studies focuses on nanocarriers to investigate drug loading and release.

Patents WO2020258584A1, CN110200941B, CN109970987A have disclosed the methods and applications of several oral nanodrugs, which encapsulate radioprotective drugs with nanomaterials to achieve drug delivery to the intestines. Although these patents achieve oral drug delivery, challenges such as low absorption efficiency and oral safety still exist, wherein the low absorption efficiency may lead to difficulties in maintaining prolonged retention and low degradation of drug in intestinal tissues. One reason is that nanomaterials are nanoscale, making it difficult to achieve long-term residence and potency in the intestines. Another reason is that the nanodrug preparation process is complicated and expensive, impeding the large-scale production and application. In addition, the adjuvants of nanodrugs are mostly chemicals with certain toxicity, such as organic reagents, and the safety of the nanodrugs has not been reported. To date, a novel gastrointestinal radioprotective drug with inexpensive price, long-term efficacy, reliable biosafety, and minimal side effects is still in demand. Therefore, the research and development of related drugs and formulations has become a top priority in the research of radioprotective drugs.

### SUMMARY

On the basis of the existing deficiency of current technology, the aim of the present invention is to provide a pharmaceutical complex for intestinal radioprotection, along with its preparation method and applications. This pharmaceutical complex overcomes the above mentioned limitations of drugs for radioprotection, such as low absorption efficiency and poor oral safety, enabling long-term retention and slow degradation of AMF in intestinal tissues. This enhances the local protective effect of the drug on intestinal tissues, preventing radiation-induced intestinal injury from radiotherapy for abdominal/pelvic tumors or from low-dose radiation from the environment, while avoiding the systemic toxic side effects related with intravenous administration. The preparation process is simple, practicable, and scalable. This pharmaceutical complex can also supply a small number of proteins, unsaturated fatty acids, and trace elements required for the human body.

The first purpose of this invention is to provide a pharmaceutical complex for radioprotection that combines AMF and natural microalgae, wherein both the natural microalgae and the pharmaceutical complex are micrometer sized. Within the pharmaceutical complex, the mass ratio of natural microalgae to Amifostine ranges from 1:0.5 to 1:8 (i.e., SP:AMF from 1:0.5 to 1:8). The binding between AMF and natural microalgae is driven by osmotic pressure. Under the osmotic pressure of different solutions inside and outside the microalgae, AMF molecules can be combined to the surface of the microalgae or enter the internal through the water channel pores of the microalgae surface to form a pharmaceutical complex. The osmotic pressure-driven binding between AMF and natural microalgae in the pharmaceutical complex can be confirmed by infrared detection where the pharmaceutical complex showed characteristic peaks at 758 and 3302 cm⁻¹ under infrared scanning in the range of 400 to 4000 cm⁻¹.

Preferably, the pharmaceutical complex comprises a solvent.

Preferably, the solvent is selected from one or more of the following: sterile phosphate buffer solution, ultrapure water, distilled water, or saline.

Preferably, the pharmaceutical complex has prolonged intestinal retention and slow degradation properties, which implies that the complex can still be detected in the intestines for more than 8 hours after administration, 4 hours prior to irradiation. preferably, the detection method comprises administering a certain dose to Balb/c nude mice following a 12-hour fasting period by gavage, with fluorescence imaging revealing the localization of the pharmaceutical complex within the small intestine of the mice. Preferably, the dose of the complex ranges from 120-600 mg/kg, with a preferred dose of 360 mg/kg. Furthermore, the preferred duration is 24 hours.

More preferably, the fluorescence imaging technique utilizes Cy5.5 channel with an excitation wavelength of 605 nm and an emission wavelength of 615-665 nm in the chlorophyll channel.

Preferably, the pharmaceutical complex exhibits considerable oral safety, which is assessed through a method comprising a daily oral administration of a specific dose to the Balb/c mice, followed by the body weight of the mice remaining unchanged, and the hematological parameters as well as liver and kidney functions remain within normal ranges. Preferably, the certain dose of complex is 120-600 mg/kg, with a more preferred dose of 360 mg/kg. Preferably, the preferred duration of continuous administration is 30 days.

Preferably, the natural microalgae is Spirulina platensis.

Preferably, the length of the Spirulina platensis is between 100-500 μm.

The second purpose of this invention is to provide preparation method of the pharmaceutical complex for radioprotection. The preparation method entails the preparation of an AMF solution, natural microalgae, and the pharmaceutical complex. The method comprises the following steps:

Preparation of natural microalgae powder and Amifostine solution: Micrometer-sized cultured microalgae are centrifuged to remove the supernatant. Collecting the precipitate after washing and obtaining microalgae powder through post-processing. Weigh the solid AMF to prepare an AMF solution with a concentration of 0.04 to 0.48 mg/ml.

Preferably, the centrifugation speed and time are 4500 rpm and 10 minutes, respectively.

Preferably, the solution used for washing and precipitating is selected from at least one of sterile phosphate buffer solution or distilled water.

Preferably, the number of washing and precipitating cycles is 3 to 5.

Preferably, the AMF solution is mixed with at least one of phosphate-buffered saline solution or distilled water.

### (2) Pharmaceutical Complex Preparation

The pharmaceutical complex is prepared by mixing microalgae powder and AMF solution in a feed ratio of 1:0.8 to 1:10.

Preferably, the mixing preparation comprises adding microalgae powder to the AMF solution under specific temperature and dark conditions, followed by stirring, centrifugation, collection of the precipitate, washing 3-5 times, and post-processing to obtain the solid powder of the pharmaceutical complex.

Preferably, the specific temperature is 2-8° C.

Preferably, the stirring speed is 60-200 rpm.

Preferably, the stirring time is 6-12 hours.

The third purpose of this invention is to provide a pharmaceutical composition that includes at least one active component and at least one pharmaceutically acceptable additive. The active component is selective from the pharmaceutical complexes prepared by the above method or the pharmaceutical complex itself.

Preferably, additives include traditional diluents, excipients, fillers, adhesives, wetting agents, disintegrators, absorption enhancers, surfactants, adsorption carriers, lubricants, and similar substances; and when necessary, flavorings and sweeteners, and the like.

The fourth purpose of this invention is to provide the use of the pharmaceutical complex, the pharmaceutical complex obtained by the preparation method, or the pharmaceutical composition in the preparation of drugs for the protection against cancer treatment-related damages.

Preferably, the tumor is selected from at least one type of solid tumor.

Preferably, the solid tumor is selected intestinal tissues-related tumors.

Preferably, at least one type of abdominal or pelvic solid tumors.

Preferably, the abdominal or pelvic solid tumor is selected from among pancreatic cancer, prostate cancer, and colon cancer; further, colon cancer is most preferred.

Preferably, after entering the intestine by oral administration, the pharmaceutical complex or composition dissolves with digestion, slowly releasing the medication. This ensures that the pharmaceutical complex is fully covered and distributed throughout the proximal, medium, and distal small intestine. This dramatically increases the drug concentration in small intestinal tissues, effectively protecting the small intestine while decreasing the drug concentration in the blood, thereby preventing systemic toxicity.

Preferably, the pharmaceutical complex or composition does not interfere with the killing/inhibitory effect of X-rays on tumor tissues during radiotherapy for cecal in situ colon cancer.

Preferably, the pharmaceutical complex or composition is applicated for long-term oral radioprotection in clinical settings, displaying high biosafety, effectively avoiding the potential toxic side effects of AMF, and being suitable for daily, long-term oral use.

Preferably, the oral pharmaceutical complex or composition for intestinal radiation protection overcomes the drawbacks of the singular mode of Amifostine intravenous injection and the insufficient efficacy of nanomedicine compounds in intestinal tissues.

The fifth purpose of this invention is to provide the use of a pharmaceutical composition in intestinal regulation.

Preferably, the intestinal regulation includes at least one of inflammation regulation or the supplementation of intestinal nutrition.

Preferably, the nutritional components are selected from proteins, unsaturated fatty acids, carotenoids, vitamins, and a variety of trace elements such as iron, iodine, zinc, or polysaccharides among probiotics.

The sixth purpose of this invention is to provide an oral formulation. The active ingredient of the oral formulation comprises at least one of the pharmaceutical complex, the pharmaceutical complex obtained by the preparation method, or the pharmaceutical composition, along with at least one pharmaceutically acceptable carrier or additive.

Preferably, the formulation is a solid formulation.

Preferably, the solid formulation is selected from tablets, powders, granules, or capsules; each form can be prepared according to conventional methods in the field of pharmacy.

Preferably, the additives comprise traditional diluents, excipients, fillers, adhesives, wetting agents, disintegrators, absorption enhancers, surfactants, adsorption carriers, lubricants, and similar substances; and when necessary, flavorings and sweeteners, and the like.

### Technical Benefits

1. For the first time, this invention has prepared a pharmaceutical complex that features osmotic pressure-driven binding between the radioprotective drug Amifostine and natural microalgae in a specific mass ratio (SP:AMF=1:0.5 to 1:8). Under the range of 400-4000 cm⁻¹ infrared scanning, this pharmaceutical complex exhibits characteristic peaks at 758 and 3302 cm⁻¹. The pharmaceutical complex breaks the singular mode of intravenous injection of the Amifostine, maintaining high levels of drug activity and oral safety after administration, enabling prolonged intestinal retention and slow degradation in intestinal tissues.
2. This invention breaks through the limitations that Amifostine cannot provide comprehensive radioprotection to intestinal tissues. After application, this pharmaceutical complex can thoroughly cover and distribute throughout the proximal, medial, and distal small intestine, which significantly increases the drug concentration in small intestinal tissues, fully exerting its protective effect on small intestinal tissues and cells; simultaneously, the pharmaceutical complex lowers the drug concentration in the blood, avoiding systemic toxicity and making it suitable for long-term radiotherapy.
3. The pharmaceutical complex in this invention, characterized by osmotic pressure-driven binding peaks (detected by FTIR), is a micrometer-scale complex. Compared to nanodrug formulations or complexes, the pharmaceutical complex in this invention prolongs radioprotection and regulates intestinal inflammation; after application, the pharmaceutical complex exhibits gradual fragmentation and degradation, indicating ample release of Amifostine. Meanwhile, the piecemeal degradation of the pharmaceutical complex also demonstrates its degradability by the digestive system, avoiding the toxic side effects resulted from long-term retention in the body.

In this invention, the term “SP” refers to Spirulina platensis.

In this invention, the term “AMF” refers to the intestinal radiation protection drug Amifostine.

In this invention, the term “SP@AMF” refers to the pharmaceutical complex.

In this invention, the term “SGF” refers to simulated gastric fluid.

### BRIEF DESCRIPTION OF THE DRAWINGS

FIG. 1. Lyophilized powder and PBS suspension of the prepared SP@AMF, and its bright-field microscope, fluorescence and SEM images. Scale bar=20 μm.

FIG. 2. The positive correlation between the mass ratio of SP/AMF during preparation and the mass ratio of SP/AMF in the pharmaceutical complex, the corresponding formula is y=1.3820x−0.0427, R²=0.9983, wherein x is the mass ration of SP/AMF during preparation, y refers to the mass ratio of SP/AMF in the pharmaceutical complex.

FIG. 3. Fourier transform infrared (FTIR) spectra of the pharmaceutical complex (SP@AMF), Amifostine (AMF) and Spirulina platensis (SP).

FIG. 4. Release profiles of AMF from SP@AMF after being treated with simulated gastric fluid (SGF) for 1 and 2 h. Y axis illustrates the percentage of cumulative released drugs in total loaded drugs. Untreated SP@AMF was used as control (n=3 independent experiments). The data show means+SD.

FIG. 5. pH values of the SGF supernatant containing different concentrations of SP@AMF (n=3 independent experiments). The data show means+SD. P was calculated using two-tailed t-test.

FIG. 6. Fluorescence images of in vivo biodistribution of the pharmaceutical complex (SP@AMF) at different time points (0-24 h) after oral administration.

FIG. 7. Scanning electron microscope (SEM) images (pseudo-color) of the pharmaceutical complex between the intestinal villi. Scale bar=20 μm.

FIG. 8. SEM images of the pharmaceutical complex (SP@AMF) in the stomach, small intestines, and large intestines 4 h after oral administration. Scale bar=20 μm.

FIG. 9. Immunohistochemistry (IHC) images of the regenerating crypts in the small intestine (duodenum, jejunum, and ileum) stained by Ki67 at day 3 after being treated by sham irradiation+PBS (PBS group), 12 Gy abdominal X-ray (IR)+PBS, IR+SP, IR+AMF, and IR+SP@AMF (n=6 biologically independent animals). The black dotted line indicates the Ki67-stained regenerating crypts. Scale bar=100 μm.

FIG. 10. Represented IHC images of the small intestine (duodenum, jejunum, and ileum) stained by Masson Trichrome at day 30 after being treated by sham irradiation+PBS (PBS group), 12 Gy abdominal X-ray (IR)+PBS, IR+SP, IR+AMF, and IR+SP@AMF. The blue areas show fibrosis formation. Scale bar=100 μm.

FIG. 11. SP@AMF downregulates pro-inflammatory cytokine IL-10 level in the small intestine tissue (n=6 biologically independent animals). The data show means+SD. P was calculated using two-tailed t-test. *P versus IR+PBS group (*<0.05, **<0.01, ***<0.001, n.s., no significance). Red * represents P value between IR+AMF group and IR+SP@AMF group.

FIG. 12. SP@AMF downregulates pro-inflammatory cytokine IL-6 level in the small intestine tissue (n=6 biologically independent animals). The data show means+SD. P was calculated using two-tailed t-test. *P versus IR+PBS group (*<0.05, **<0.01, ***<0.001, n.s., no significance). Red * represents P value between IR+AMF group and IR+SP@AMF group.

FIG. 13. SP@AMF downregulates pro-inflammatory cytokine TNF-α level in small intestine tissue (n=6 biologically independent animals). The data show means+SD. P was calculated using two-tailed t-test. *P versus IR+PBS group (*<0.05, **<0.01, ***<0.001, n.s., no significance). Red * represents P value between IR+AMF group and IR+SP@AMF group.

FIG. 14. The tumor volume and weight in nude mice with cecal tumor in situ after abdominal radiation therapy (*, p value<0.05; **, p value<0.01; ***, p value<0.001).

FIG. 15. Hematological tests and serum biochemicals tests of the mice after the daily administration of PBS, SP, AMF, or SP@AMF for 30 days (n=5 biologically independent animals). The data show means±SD. P was calculated using two-tailed t-test. WBC white blood cells, MCH mean corpuscular hemoglobin, AST aspartate transferase, CREA creatinine.

FIG. 16. Body weight of mice monitored for 30 days of continuous oral administration of pharmaceutical complexes (n=5 biologically independent animals). The data shows means±SD. P was calculated using two-tailed t-test (*, p value<0.05; **, p value<0.01; ***, p value<0.001).

### DETAILED DESCRIPTION OF THE INVENTION

The invention is further described with the following drawings and examples, but the invention is not limited to the following examples.

### Example 1: Synthesis of SP@AMF

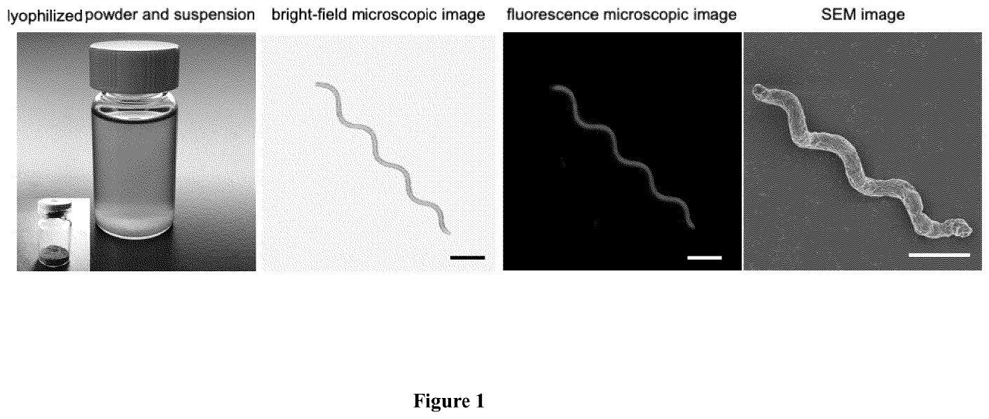

Spirulina platensis (SP) was cultured under sterile conditions, with the suspension of SP centrifuged (4500 rpm, 10 min). The supernatant was discarded, and the pellet was washed three times with phosphate-buffered saline (PBS) to remove residual culture medium, which was then freeze-dried to obtain a solid SP powder. A PBS solution containing 3.125 mg/mL Amifostine (AMF) was prepared. Based on a mass ratio of SP:AMF of 1:0.6, the aforementioned SP powder was added and thoroughly dispersed in a 50 ml sterile centrifuge tube wrapped in tin foil, followed by gently shaken (60 rpm) on a shaker at 4° C. for 12 hours. The supernatant was discarded after centrifugation, while the precipitate was collected after washing three times, followed by being lyophilized to obtain a solid powder of the pharmaceutical complex SP@AMF (FIG. 1). The lyophilized powder of SP@AMF was stored in the dark, sealed, dry, at 2-8° C. conditions. This preparation yielded a pharmaceutical complex with an SP:AMF mass ratio of 1:1.25. The drug complex powder collected after post-processing was observed under a microscope and scanning electron microscopy (SEM), demonstrating the uniform liquid (distilled water) suspensions of SP@AMF powders at different mass ratios, with a 3D spiral shape and red fluorescence imaging characteristics (FIG. 1).

Following the SP@AMF preparation method described above, the pharmaceutical complex with different mass ratios of SP/AMF (SP:AMF=1:0.8-1:10, i.e., SP/AMF=0.1-1.25) were prepared, where the morphology under SEM was the same as that of the pharmaceutical complex with an SP:AMF mass ratio of 1:1.25, all being 3D spiral-shaped and having red fluorescence imaging characteristics. The infrared spectra of SP@AMF at different mass ratios (SP:AMF=1:0.5-1:8) showed characteristic peaks of both SP (1541, 1654, and 2926 cm⁻¹) and AMF (587, 956, and 1012 cm⁻¹); additionally, SP@AMF formed unique characteristic peaks (758 and 3302 cm⁻¹).

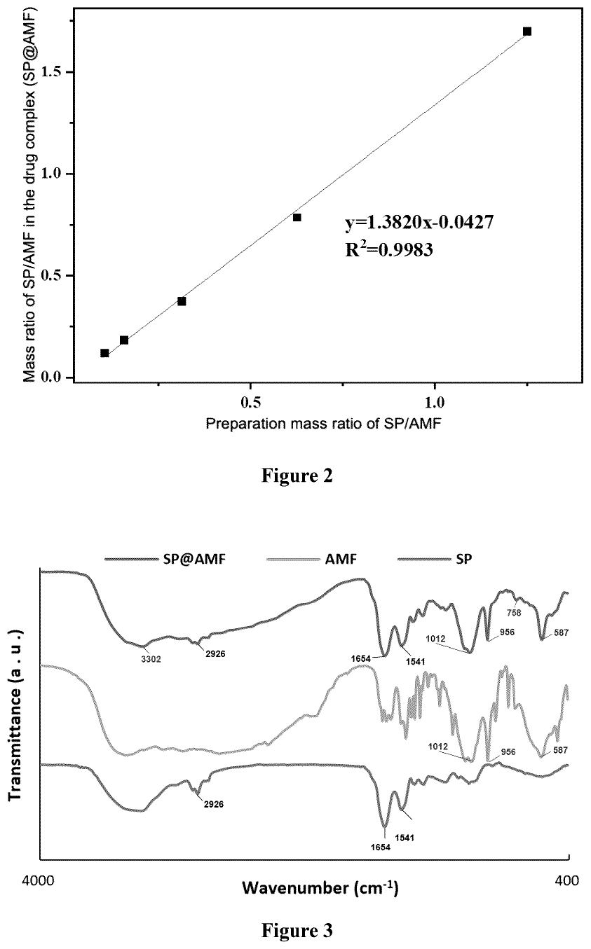

Moreover, due to the limitations of the intrinsic properties and loading capacity of SP, the relationship between SP/AMF feed ratios and the pharmaceutical complex's SP/AMF mass ratios is shown in Table 1, indicating a positive correlation between the SP/AMF feed ratios during preparation and the SP/AMF mass ratios of the resulting pharmaceutical complex. As shown in FIG. 2, x is the SP/AMF feed ratio during preparation, y is the SP/AMF mass ratio of the synthesized pharmaceutical complex, and R² is the correlation coefficient. The value of R2 closer to 1 indicates a higher degree of agreement between the experimental data and the fitting function; in this invention, R² is 0.9983, indicating a high degree of agreement between the experimental data and the fitting function.

Considering the loading efficiency and preparation cost, the pharmaceutical complex with an SP:AMF mass ratio of 1:1.25 is preferred to prepare using a mixing system with a feed ratio of SP:AMF≈1:1.7 (i.e., SP/AMF=0.6, as shown in FIG. 2). Owing to the optimal AMF loading capacity, the pharmaceutical complex with this mass ratio was used for subsequent efficacy experiments.

Additionally, Examples 2-7 and comparative examples use the pharmaceutical complex with an SP:AMF mass ratio of 1:1.25 for experiments, with other mass ratios of the pharmaceutical complex also applicable in this invention.

TABLE 1
The corresponding relationship between the SP/AMF feed ratio
and the mass ratio of SP/AMF in the pharmaceutical complex

| SP/AMF | mass ratio of SP/AMF in the | mass ratio of SP/AMF in |
|---|---|---|
| feed ratio | pharmaceutical complex (mean) | the pharmaceutical complex |
| 1.478 | 2.0 | 1:0.5 |
| 1.25 | 1.703 | 1:0.587 |
| 0.625 | 0.786 | 1:1.272 |
| 0.3125 | 0.376 | 1:2.660 |
| 0.1563 | 0.185 | 1:5.045 |
| 0.1213 | 0.125 | 1:8 |

### Example 2. Verification of Drug Loading Performance

The infrared spectra of SP@AMF, SP and AMF were detected using Fourier transform infrared spectroscopy (FTIR) in the range of 400-4000 cm⁻¹ (FIG. 3), which elucidated the characteristic peaks of SP at 1541, 1654, and 2926 cm⁻¹, and the characteristic peaks of AMF at 587, 956, and 1012 cm⁻¹. The infrared spectrum of SP@AMF exhibited the characteristic peaks of both SP (1541, 1654, and 2926 cm⁻¹) and AMF (587, 956, and 1012 cm⁻¹); moreover, SP@AMF formed its unique characteristic peaks (758 and 3302 cm⁻¹). The above results confirm the successful loading of AMF into SP.

### Example 3. In Vitro Drug Release Performance Testing

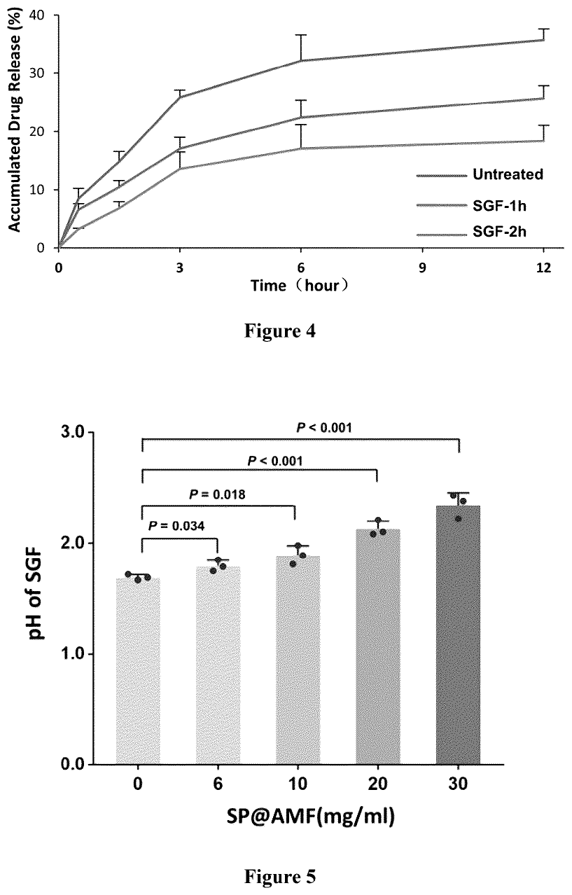

1 mg of the synthesised pharmaceutical complex was added to 1 ml of simulated gastric fluid (SGF) and shaken (180 rpm) at 37° C. for 0, 1, and 2 hours, respectively. After centrifugation to remove the supernatant, the precipitate was transferred to 5 ml of phosphate buffered saline (PBS) and shaken (180 rpm) at 37° C. At 0.5, 1.5, 3, 6, 12, and 24 hours after transfusion, the concentration of AMF in the supernatant was measured and the in vitro release curve of AMF was plotted (FIG. 4). The in vitro release curve demonstrated prolonged release of AMF from the pharmaceutical complex. Even after 1-2 hours of pretreatment with simulated gastric fluid, the pharmaceutical complex could still guarantee a drug release of over 50%, indicating that SP can protect most AMF to enter the intestine and gradually release AMF. Additionally, by testing the pH value of simulated gastric fluid containing different concentrations of the pharmaceutical complex (FIG. 5), the SP@AMF pharmaceutical complex was proven with certain capabilities to neutralize gastric fluid acidity, in favor of protecting the activity of drugs.

### Example 4. Detection of In Vivo Distribution and Degradation after Oral Administration

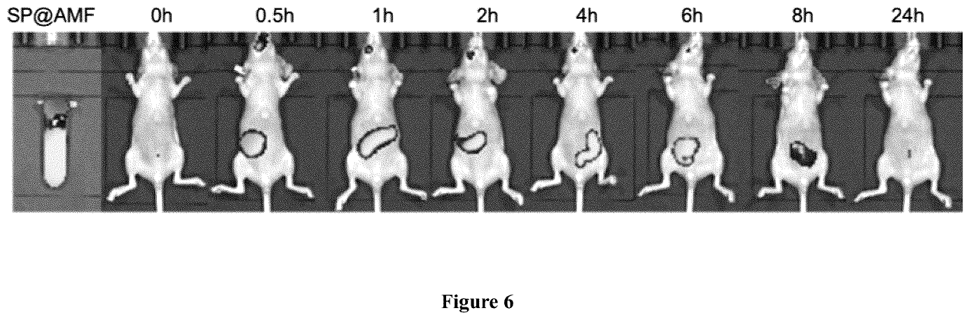

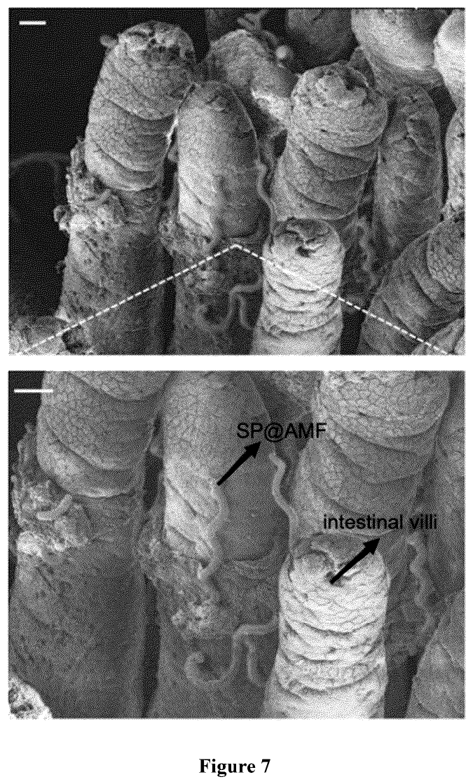

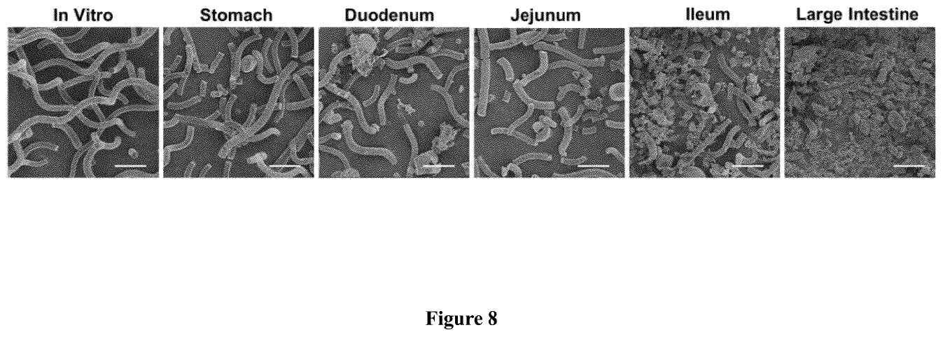

In virtue of the auto-fluorescence of the chlorophyll contained in SP, the in vivo distribution pattern after oral administration was monitored with a live imaging system. The pharmaceutical complex was resuspended in distilled water and administered orally to Balb/c nude mice after fasting for 12 h at a dose of 360 mg/kg SP@AMF (containing approximately 200 mg/kg AMF). Mice were anaesthetised at 0, 0.5, 1, 2, 4, 6, 8, 24 hours after gavage, followed by fluorescence imaging in the chlorophyll channel of SP (channel Cy5.5, excitation wavelength 605 nm, emission wavelength 615-665 nm) (FIG. 6). Three to four hours after oral administration of SP@AMF, the middle segment of the small intestine was taken and mildly washed of contents, where the inner surface of the small intestine was flattened, and prepared for SEM to observe the morphology of SP@AMF between the intestinal villi (FIG. 7). Meanwhile, the contents from different segments of the digestive tract were removed, lightly washed, and the morphological changes of SP@AMF were observed with scanning electron microscopy (FIG. 8). According to the results, the fluorescence of SP@AMF was consistently concentrated in the abdomen with a high fluorescence intensity within 0-6 hours after oral administration, indicating a long retention time in the intestines, which further promoted the accumulation of drug concentration in intestinal tissues. Due to the spiral shape and similar length to that of intestinal villi, the pharmaceutical complex was widely distributed between the intestinal villi with direct contact, which is beneficial for better drug absorption by intestinal epithelial cells (FIG. 7). According to FIG. 8, from the proximal (stomach) to the distal (large intestine) parts of the digestive tract, the pharmaceutical complex exhibited a pattern of gradual fragmentation and degradation, not only beneficial for fully release of AMF but also hinting that the pharmaceutical complex can be easily degraded by the digestive system, avoiding long-term retention in the body.

### Example 5. Protective Effect of the Pharmaceutical Complex Against Intestinal Radiation Damage

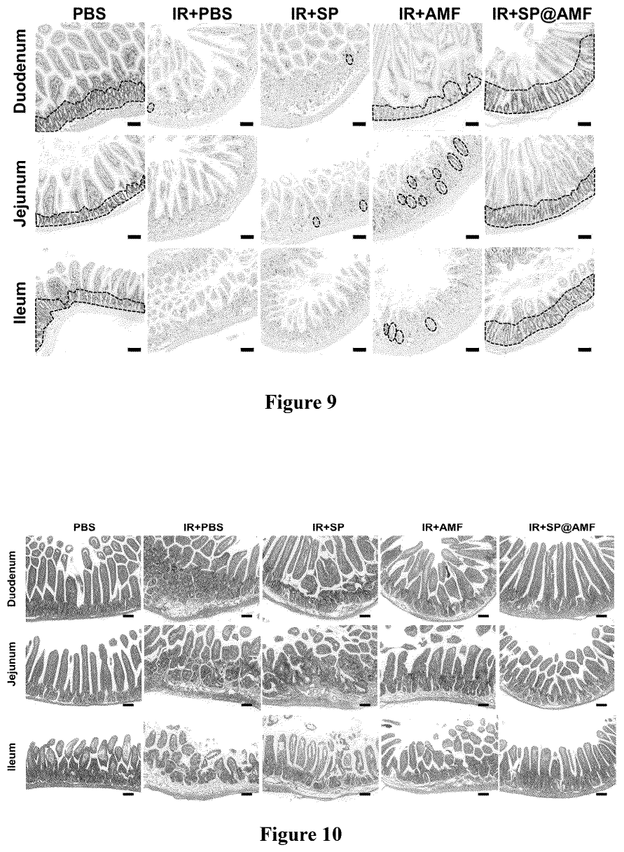

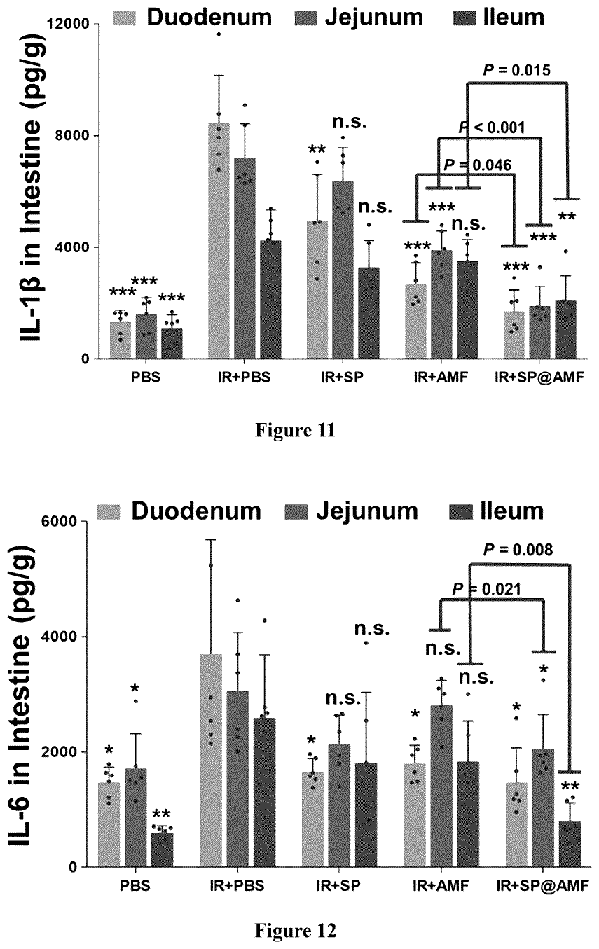

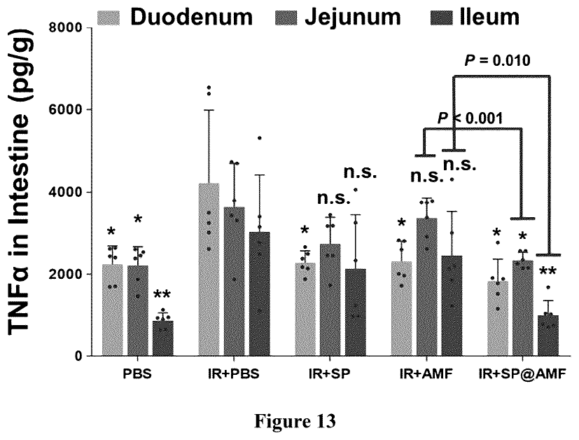

According to the distribution pattern of SP@AMF in the digestive system, approximately four hours after oral administration, SP@AMF could achieve comprehensive coverage and drug distribution in the small intestine. Therefore, the animals were subjected to abdominal X-ray irradiation and the protective effect of the drug against X-ray-induced intestinal damage was assessed by pathological examination four hours after oral administration of SP@AMF. The pharmaceutical complex was resuspended in distilled water and administered via gavage to fasted Balb/c white mice at a dose of 360 mg/kg SP@AMF (containing approximately 200 mg/kg AMF). Four hours after gavage, the animals were anaesthetized and subjected to abdominal X-ray irradiation (12 Gydose rate=8.415 Gy/min). The control group received an equal volume of distilled water, SP, and AMF. The mice were euthanized three days after irradiation, and small intestinal tissues were collected for pathological sectioning and immunohistochemistry (Ki67) to determine short-term radiation damage to the small intestine (proliferative crypts) (FIG. 9 Thirty days after irradiation, small intestinal tissues were collected for pathological sectioning and Masson's trichrome staining to assess long-term radiation damage to the small intestine (fibrosis) (FIG. 10). Small intestinal tissue homogenates were prepared to measure the level of the inflammatory factors IL-1β, IL-6, and TNF-α (Elisa kit, Boster Biological Technology Co. Itd) (FIGS. 11, 12, 13). The SP@AMF group maintained normal crypt proliferative activity in the duodenum, jejunum, and ileum, with less late-stage fibrosis and lower levels of inflammatory markers. All indicators were much higher than those of other treatment groups, including the AMF group, implying that the pharmaceutical chemical significantly improved the radiatioprotective impact of AMF on intestinal tissues.

### Example 6. Effect of the Pharmaceutical Complex on Tumor Killing in Radiotherapy

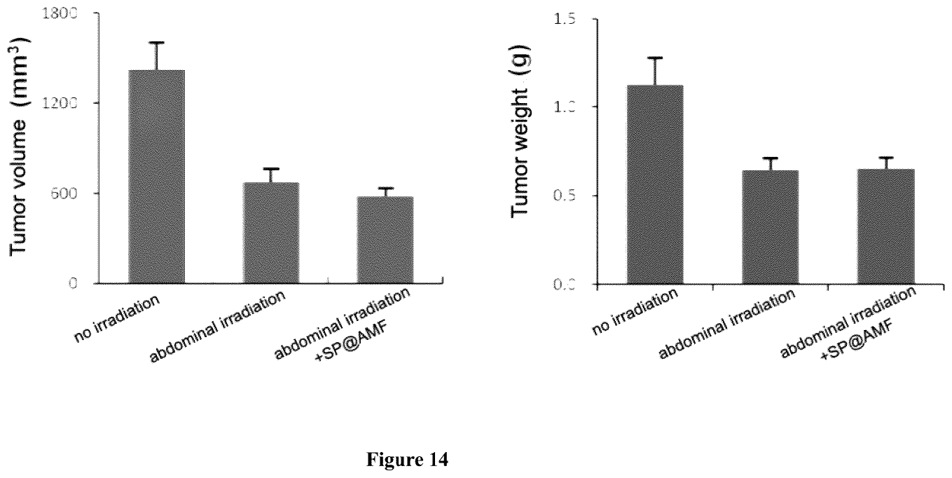

A cecal in-situ colon cancer animal model was established to further simulate the clinical process of tumor radiotherapy, followed by pre-treatment of the radioprotective pharmaceutical complex before abdominal radiotherapy. To construct the animal model, transfected CT26-luci colon cancer cells expressing luciferase were inoculated into the cecal wall of Balb/c nude mice. An animal living imaging apparatus was used to detect the tumor fluorescence signals to monitor the tumor growth. After the diameter of tumor reached 1-2 cm, the animals were fasted for 12 hours. Then the mice were fed with 360 mg/kg SP@AMF via gavage. The animals were under anaesthesia and subjected to 12 Gy abdominal X-ray irradiation after four hours. Two control groups were established: a non-irradiation group (sham irradiation+distilled water orally), and an abdominal irradiation group (abdominal X-ray irradiation of 12Gy+distilled water orally). Tumor growth was monitored weekly using an animal living imaging apparatus, and when the tumour reached a diameter of 10-12 cm, the animals were euthanized and the tumors were excised for volume measurement and weighing (FIG. 14). The results revealed no significant statistical difference in tumour volume and weight between the abdominal irradiation group and the abdominal irradiation+SP@AMF group, indicating that the use of this pharmaceutical complex had no radioprotection effects on tumour tissues and had no effect on tumor-killing effect of tumor radiotherapy.

### Example 7. Safety Test of Long-Term Oral Administration of the Pharmaceutical Complex

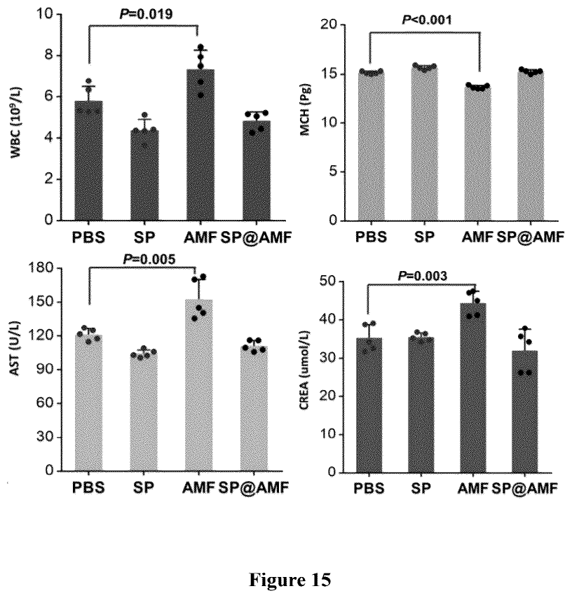

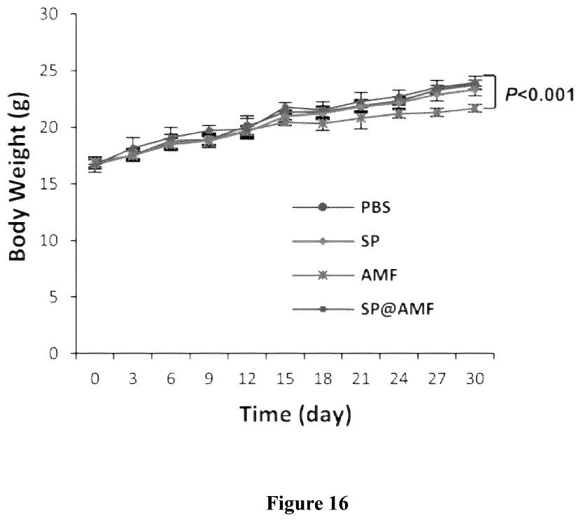

The SP@AMF was resuspended with distilled water. The Balb/c white mice were administered with a dose of 360 mg/kg SP@AMF via gavage, once daily for 30 consecutive days. Blood samples were taken for routine blood, liver, and kidney function testing (FIG. 15), and body weight changes were monitored throughout this period (FIG. 16). Control groups were given equal amounts of distilled water, SP, and AMF, respectively. According to the results, the AMF group experienced significant weight loss, hematological abnormalities, and liver and kidney dysfunction. On the contrary, administration of SP@AMF exhibits excellent biosafety, with all indications of above tests remaining normal. This could be due to the slow release of AMF by SP@AMF in the intestine, which improves the local distribution of the pharmaceutical complex in the intestines, reduces drug concentration in the blood, and thus avoids systemic toxicity caused by the wide distribution of AMF after direct oral administration.

### Comparative Examples

The experimental steps of the comparative example refer to the patent CN110200941A.

### Synthesis of Nanodrug Formulation

1. Arginine (0.867 g, 4.977 mmol) was mixed in 40 ml of morpholine ethanesulfonic acid solution (25 mM, pH 5.0). N-hydroxysuccinimide (2.291 g, 19.908 mmol) and 1-(3-dimethylaminopropyl)-3-ethylcarbodiimide hydrochloride (3.816 g, 19.908 mmol) were added successively. The solution was activated for two hours.
2. Chitosan solution dissolved in morpholine ethanesulfonic acid (1.0 g, 4.977 mmol) was added to the mixture in step 1. The mixture was constantly stirred at room temperature for 24 hours. The reaction was stopped by adding sodium hydroxide (0.1 M).
3. AMF dissolved in a mixture of water and acetonitrile (v/v=1/1, 4.5 mg/nil, 10 mL) was gently added to the polymer solution in step 2 (10 mg/ml, 100 mL). The mixture was stirred continuously with nitrogen overnight to eliminate the acetonitrile. The supernatant was then centrifuged and lyophilized.
4. The lyophilized material (20.0 mg) was mixed with a dopamine solution (2 mg/mL, 40 mL, pH 8.5) The mixture was agitated for 3 hours at room temperature before being centrifuged and rinsed with deionized water to extract the nanodrug from the supernatant. This nanodrug exhibited an AMF loading similar to the pharmaceutical complex, with an SP:AMF mass ratio of 1:1.25.

### In Vivo Radioprotection Effect Test

The above preparations were resuspended in distilled water, and then administered to fasted Balb/c mice by gavage with nanodrug containing AMF 200 mg/kg. Four hours after gavage, the animals were anaesthetized and subjected to 12Gy abdominal X-ray irradiation (dose rate=8.415 Gy/minute). The animals were sacrificed three days after irradiation and small intestinal tissues were collected for pathological sectioning and immunohistochemical staining (Ki67) to determine the extent of short-term radiation damage to the small intestine (proliferative crypts). Small intestinal tissues were taken 30 days post-irradiation for pathological sectioning and Masson trichrome staining to assess long-term radiation damage (fibrosis). Intestinal tissue homogenates were prepared to measure inflammatory factors IL-1β, IL-6, and TNF-α. Results showed that this nanodrug had some protective effects on the proliferative activity of crypts in the proximal small intestine (duodenum) (about 50% of the normal control group), but weaker protective effects on the middle and distal sections of the small intestine, jejunum, and ileum, with the number of surviving crypts only about 20% of the normal control, with similar trends in other indicators such as the degree of late-stage fibrosis and levels of 3 inflammatory factors. These findings suggest that, although the nanodrug has some radioprotective effects on radiation enteritis, its protective effect is not ideal and cannot cover the entire length of the small intestine.

The following examples and comparative examples are provided only for illustrative purposes and are not intended to limit the scope of the invention. Furthermore, it should be noted that, after reading the contents disclosed by this invention, individuals in the field can make various changes or modifications to the invention, and these equivalent forms are also within the scope indicated by the claims attached to this application.

What is claimed is:

1. A pharmaceutical complex for radioprotection, comprising amifostine and natural microalgae, wherein both the natural microalgae and the pharmaceutical complex are of micrometer scale; within the pharmaceutical complex, the mass ratio of natural microalgae to amifostine ranges from 1:0.5 to 1:8, with an osmotic pressure-driven binding between amifostine and the natural microalgae; the method for detecting the osmotic pressure-driven binding comprises infrared scanning in the range of 400-4000 cm⁻¹, wherein characteristic peaks of both Amifostine and natural microalgae are observed at 758 and 3302 cm⁻¹; the natural microalgae is Spirulina.

2. The pharmaceutical complex according to claim 1, wherein the pharmaceutical complex further comprises a solvent.

3. The pharmaceutical complex according to claim 2, wherein the solvent is selected from one or more of the following: sterile phosphate buffer solution, ultrapure water, distilled water, and saline.

4. The pharmaceutical complex according to claim 1, wherein a length of the Spirulina is between 100-500 μm.

5. A method for preparing a pharmaceutical complex for radiation protection, comprising the following steps:
(1) preparation of natural microalgae powder and an amifostine solution: culturing a micrometer-scale microalgae, centrifuging the cultured micrometer-scale microalgae to discard the supernatant; collecting the precipitate after washing, and obtaining microalgae powder through post-processing; weighting a solid amifostine and preparing an amifostine solution with a concentration of 0.05-0.5 mg/mL;
(2) preparation of the pharmaceutical complex: mixing the obtained microalgae powder and the amifostine solution in a ratio of 1:0.8-1:10 to prepare the pharmaceutical complex;
wherein the natural microalgae is Spirulina.

6. The method according to claim 5, wherein, in step (1), the centrifugation speed and time are 4500 rpm and 10 minutes, respectively.

7. The method according to claim 5, wherein, in step (1), the washing solution for the precipitate is selected from at least one of distilled water or sterile phosphate buffer solution.

8. The method according to claim 5, wherein, in step (1), the number of washings is 3-5 times.

9. The method according to claim 5, wherein, in step (1), the amifostine solution is homogeneously prepared using at least one of sterile phosphate buffer solution or distilled water.

10. The method according to claim 5, wherein, in step (2), the mixing preparation comprises adding the microalgae powder to the amifostine solution under specific temperature and light-protected conditions, followed by stirring, centrifugation, collection of the precipitate, washing 3-5 times, and post-processing to obtain the solid powder of the pharmaceutical complex.

11. The method according to claim 5, wherein, in step (2), the optimal temperature is between 2-8° C.

12. The method according to claim 5, wherein, in step (2), the stirring speed is 60-200 rpm.

13. The method according to claim 5, wherein, in step (2), the stirring time is 6-12 hours.

14. The method according to claim 5, wherein the post-treatment comprises at least one of drying or preparation of the pharmaceutical complex suspension.

15. The method according to claim 14, wherein the drying is lyophilization.

16. The method according to claim 14, wherein the solvent for suspension preparation is selected from at least one of sterile phosphate buffer solution or saline solution.

17. A pharmaceutical complex, comprising at least one active component and at least one pharmaceutically acceptable additive; the active component is selected from the pharmaceutical complex according to claim 1.

18. The pharmaceutical complex according to claim 17, wherein the additives comprise traditional diluents, excipients, fillers, adhesives, wetting agents, disintegrators, absorption enhancers, surfactants, adsorption carriers, lubricants.

19. The pharmaceutical complex according to claim 17, wherein the additives further comprise flavorings and sweeteners.
